(19) United States
(12) Patent Application Publication
(10) Pub. No.: US 2009/0163727 A1
(43) Pub. Date: Jun. 25, 2009

(54) CONVERSION 9-DIHYDRO-13-ACETYLBACCATIN III TO 10-DEACETYLBACCATIN III

(75) Inventor: Liu Jian, Fredericton (CA)

Correspondence Address: BENOIT & COTE, s.e.n.c., 1001, DE MAISONNEUVE BOULEVARD WEST, SUITE 210, MONTREAL, QC, H3A 3C8, CA

(73) Assignee: 6570763 CANADA INC., Montreal, QC (CA)

(21) Appl. No.: 12/281,716

(22) PCT Filed: Mar. 5, 2007
(86) PCT No.: PCT/CA2007/000350

§ 371 Date: Dec. 16, 2008

(30) Foreign Application Priority Data
Mar. 8, 2006 (CA) 2,538,860

(51) Int. Cl. C07D 303/40

(52) U.S. Cl. 549/510

(57) ABSTRACT

The present invention relates to a process is provided for the conversion of 9-dihydro-13-acetylbaccatin to 10-deacetylbaccatin III. The process includes four specific interrelated steps. The first step involves protecting the 7-hydroxyl group of 9-dihydro-13-acetylbaccatin and converting that 7-hydroxyl-protected 9-dihydro-13-acetylbaccatin to 7, 13-diacetyl-9-dihydrobaccatin III. The second step involves reacting that 7, 13-diacetyl-9-dihydrobaccatin III with 4-methylmorpholine N-oxide in a suitable solvent and oxidizing that reaction product to yield 7, 13-diacetylbaccatin. The third step involves deacetylating that 7, 13-diacetyl-9-dihydrobaccatin III to yield 7-acetylbaccatin III. The fourth and final step involves converting that 7-acetylbaccatin III to 10-deacetylbaccatin III.

### BACKGROUND OF THE INVENTION

(a) Field of the Invention

The present invention relates to a process for the conversion of 9-dihydro-13-acetylbaccatin to 10-deacetylbaccatin III.

(b) Description of Prior Art

Paclitaxel (taxol) is a potent antitumor compound. Paclitaxel exhibits a unique mechanism for preventing the growth of cancer cells by affecting the microtubules, which play an important role in cell division and other cell functions. At the beginning of cell division, a large number of microtubules are produced, and as the division reaches an end, the microtubules are normally broken down. Taxol prevents microtubules from breaking down, which has the effect of clogging up cancer cells to an extent that the cells cease to grow and divide.

Taxol is clinically effective for the treatment of refractory human ovarian and breast cancer, and has exhibited promising activity against a number of other types of cancers, e.g., liver, peritoneal, cervical, prostate, colon, and esophageal cancers.

Taxol was primarily extracted from the bark of the Pacific yew Taxus brevifolia. Unfortunately, the yew grows very slowly, approximately eight inches per year, and therefore the tree is a limited source of taxol. This has lead researchers to seek alternative means for producing taxol and analogs thereof which may display superior antitumor activity.

Many taxanes, e.g. paclitaxel and docetaxol are being aggressively studied and tested for use as cancer treating agents. As described in many publications, e.g., Canadian Patent Application No. 2,188,190, published Apr. 18, 1998 in the name of Zamir et al, the taxanes are active in various tumor systems. Taxanes are substances occurring naturally in yew trees, e.g., Taxus canadensis, which is common in Eastern Canada and the United States. One of the chemicals extracted from the needles of Taxus canadensis is 9-dihydro-13-acetylbaccatin III, which is used to produce, inter alia, 10-deacetylbaccatin III, which is a useful intermediate for the preparation of paclitaxel and analogues thereof.

U.S. Pat. No. 6,197,987, patented Mar. 6, 2001, by Liu, provided a process for preparing a taxane by oxidizing the C-9 position of 9-dihydro-13-acetylbaccatin III with a suitable oxidizing reagent, e.g., tetra-n-propylammonium perruthenate, Collin's reagent or activated methyl sulfoxide.

U.S. Pat. No. 6,812,356, patented Nov. 2, 2004, by Findlay et al, provided a process for converting 9-dihydro-13-acetylbaccatin III into, inter alia, 10-decetylbaccatin III. The 9-dihydro-13-acetylbaccatin III was converted into the 10-deacetylbaccatin III by a three step process involving (a) replacement of the C-7 hydroxyl group of the 9-dihydro compound with a protecting group, (b) oxidizing the C-7 protected compound to produce a C-9 ketone, and (c) deprotecting the C-9 ketone to produce 10-deacetylbaccatin III.

Canadian Patent Application No. 2,203,844, published October 1998, also described a process of converting 9-dihydro-13-acetylbaccatin III into 10-deacetylbaccatin III.

While, as indicated above, many processes have been proposed converting 9-dihydro-13-acetylbaccatin III into 10-deacetylbaccatin III, it has been found that such processes result in poor yields of the desired product. Thus, a need still exists for an efficient method for converting 9-dihydro-13-acetylbaccatin III (9-DHABB) to 10-deacetylbaccatin III (DAB III).

### SUMMARY OF THE INVENTION

In accordance with one embodiment of the present invention there is provided a process for the conversion of 9-dihydro-13-acetylbaccatin to 10-deacetylbaccatin III, comprising the steps of:

a) protecting the 7-hydroxl group of 9-dihydro-13-acetylbaccatin and converting the 7-hydroxyl-protected 9-dihydro-13-acetylbaccatin to 7, 13-diacetyl-9-dihydrobaccatin III.;

b) reacting the 7, 13-diacetyl-9-dihydrobaccatin III with 4-methylmorpholine N-oxide in a suitable solvent and oxidizing the reaction product to yield 7, 13-diacetylbaccatin III;

c) deacetylating the 7, 13-diacetyl-9-dihydrobaccatin III to yield 7-acetylbaccatin III; and

d) converting the 7-acetylbaccatin III to 10-deacetylbaccatin III.

In accordance with another embodiment of the present invention there is provided a process for the conversion of 9-dihydro-13-acetylbaccatin to 10-deacetylbaccatin III, comprising the steps of:

a) reacting 9-dihydro-13-acetylbaccatin with tetrabutylammonium iodide and acetyl chloride in a suitable solvent to yield 7, 13-diacetyl-9-dihydrobaccatin III.;

b) reacting the 7, 13-diacetyl-9-dihydrobaccatin III with 4-methylmorpholine N-oxide in a suitable solvent and oxidizing the reaction product to yield 7, 13-diacetylbaccatin III.;

c) deacetylating the 7, 13-diacetyl9-dihydrobaccatin III to yield 7-acetylbaccatin III; and

d) converting the 7-acetylbaccatin III to 10-deacetylbaccatin III.

In step a) the 7-hydroxl group of 9-dihydro-13-acetylbaccatin may be protected by reaction with a compound which is selected from the group consisting of acetic anhydrite, halogen-substituted acetic anhydrite, halogen-substituted acetyl chloride, acetyl bromide, a methoxybenzyl group, a tosyl group, a substituted benzyl group, dihydropyran, benzylformate, a substituted benzylformate, a methoxymethyl group, benzoylmethyl and a substituted benzoylmethyl.

7-hydroxl group of 9-dihydro-13-acetylbaccatin may be protected by reaction with acetic anhydrite.

A suitable solvent may be dichloromethane for step a) or b), acetonitrile for step b) and ethanol for step d).

In step b) the oxidizing of the reaction product of 7,13-diacetyl-9-dihydrobaccatin III with 4-methylmorpholine N-oxide in a suitable solvent may be effected with an oxidizing agent which is selected from the group consisting of tetra-n-propylammonium perruthenate, Collin's reagent and activated methyl sulfoxide.

The oxidizing agent may be tetra-n-propylammonium perruthenate.

In step c) the deacetylating of the 7,13-diacetyl-9-dihydrobaccatin III to yield 7-acetylbaccatin III may be effected with methyllithium in an ether solvent or with butyllithium in an ether solvent.

In the step d), the convertion of the 7-acetylbaccatin III to 10-deacetylbaccatin III may effected by reaction with hydrazine hydrate in a suitable solvent.

In step d), the converting of the 7-acetylbaccatin III to 10-deacetylbaccatin III may be effected with an alkali metal methoxide in a suitable solvent. The alkali metal methoxide may be sodium methoxide.

Other suitable solvents for step d) include tetrahydrofuran and dichloromethane.

In accordance with another embodiment of the present invention there is provided a process for the conversion of 9-dihydro-13-acetylbaccatin to 10-deacetylbaccatin III, which comprises reacting 7-chloroacetylbaccatin with hydrazine hydrate in a suitable solvent, which may be ethanol.

### DETAILED DESCRIPTION OF THE INVENTION

In a first aspect of the present invention, a process is provided for preparing 10-deacetylbaccatin III comprising the steps of: protecting the 7-hydroxl group of 9-dihydro-13-acetylbaccatin and converting that 7-hydroxyl-protected 9-dihydro-13-acetylbaccatin to 7, 13-diacetyl-9-dihydrobaccatin III; reacting that 7, 13-diacetyl-9-dihydrobaccatin III with 4-methylmorpholine N-oxide in a suitable solvent and oxidizing that reaction product to yield 7, 13-diacetylbaccatin III; deacetylating that 3-diacetyl-9-dihydrobaccatin III to yield 7-acetylbaccatin III; and converting that 3-diacetylbaccatin III to 10-deacetylbaccatin III.

In a second aspect of the present invention, a process is provided for preparing 10-deacetylbaccatin III comprising the steps of: reacting 9-dihydro-13-acetylbaccatin with tetrabutylammonium iodide and acetyl chloride to yield 7, 13-diacetyl-9-dihydrobaccatin III; reacting that 7, 13-diacetyl-9-dihydrobaccatin III with 4-methylmorpholine N-oxide in a suitable solvent and oxidizing that reaction product to yield 7, 13-diacetylbaccatin III; deacetylating that 7, 13-diacetyl-9-dihydrobaccatin III to yield 7-acetylbaccatin III; and converting that 7-acetylbaccatin III to 10-deacetylbaccatin III.

In a third aspect of the present invention, a process is provided for the conversion of 9-dihydro-13-acetylbaccatin to 10-deacetylbaccatin III, comprising reacting 7-chloroacetylbaccatin with hydrazine hydrate in a suitable solvent.

By a first feature of the first aspect of the present invention, in the first step, the 7-hydroxl group of 9-dihydro-13-acetylbaccatin is protected by reaction with a compound which is selected from the group consisting of acetic anhydrite, halogen-substituted acetic anhydrite, acetyl chloride, halogen-substituted acetyl chloride, acetyl bromide, a methoxybenzyl group, a tosyl group, a substituted benzyl group, dihydropyran, benzylformate, substituted benzylformate, methoxymethyl group, a benzoylmethyl group and a substituted benzoylmethyl group, but preferably by reaction with acetic anhydrite.

By a first preferred feature of the first and second aspects of the present invention, in the second step, the oxidizing of the reaction product of 7, 13-diacetyl-9-dihydrobaccatin III with 4-methylmorpholine N-oxide in a suitable solvent is effected with an oxidizing agent which is selected from the group consisting of tetra-n-propylammonium perruthenate, Collin's reagent and activated methyl sulfoxide, but preferably with tetra-n-propylammonium perruthenate, and preferably wherein the suitable solvent is dichloromethane or acetonitrile

By a second preferred feature of the first and second aspects of the present invention, in the third step, the deacetylating of the 7, 13-diacetyl-9-dihydrobaccatin III to yield 7-acetylbaccatin III is effected with methyllithium in an ether solvent or butyllithium in an ether solvent.

By a third preferred feature of the first and second aspects of the present invention, in the fourth step, the converting of the 7-acetylbaccatin III to 10-deacetylbaccatin III is effected with by reaction with hydrazine hydrate in a suitable solvent, preferably ethanol.

By a fourth preferred feature of the first and second aspects of the present invention, in the fourth step, the converting of the 7-acetylbaccatin III to 10-deacetylbaccatin III is effected by reaction with an alkali metal methoxide, preferably with sodium methoxide, in a suitable solvent, preferably in tetrahydrofuran or in dichloromethane.

By first feature of the third aspect of the present invention, the solvent is ethanol.

The starting material, 9-dihydro-13-acetylbaccatin III, can be obtained by various means including by extraction of Taxus species as described in Canadian Patent Application No. 2,203,844 published in October 1998. Briefly, as described in that patent application, the isolation process entails collecting plant material, e.g., stems and needles, and grinding and extracting the material with methanol. The extraction is carried through for about 24 hours, and the resulting mixture is filtered and the extract is collected. The extract is concentrated to about 10% of its original volume by evaporation, and further diluted with water. The aqueous solution is extracted several times with hexane to give an aqueous layer and a non-aqueous layer. The aqueous layer is extracted several times with chloroform or dichloromethane. The chloroform or dichloromethane extract is concentrated to dryness, and the residue is dissolved in a mixture of chloroform, methanol and acetone (10:1:0.5), and fractionated by dry column chromatography to obtain fractions of taxol and 9-dihydro-13-acetylbaccatin III. The fractions are combined, extracted and the 9-dihydro-13-acetylbaccatin III is crystallized out.

The following illustrates a chemical reaction flow chart for the chemical process to convert 9-DHABB (9-dihydro-13-acetylbaccatin III) to 10-DAB (10-deacetylbaccatin III).

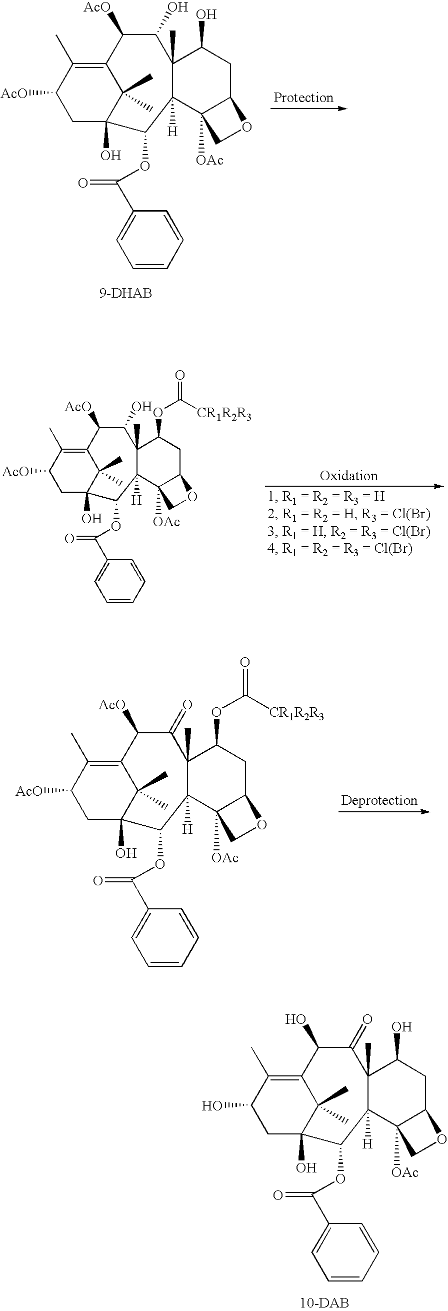

The present invention will be more readily understood by referring to the following examples which are given to illustrate the invention rather than to limit its scope.

### EXAMPLE 1

### Conversion 9-dihydro-13-acetylbaccatin III to 10-deacetylbaccatin III

1. Purification of Crude 9-Dihydro-13-acetylbaccatin III (9-DHAB)

Crude 9-DHAB (9-dihydro-13-acetylbaccatin III) was placed into a round bottom flask and 5-10 times methanol was added, and the mixture was refluxed for 1 hour or until all 9-DHAB were dissolved. Some yellow solid, which is insoluble in methanol, was filtered out. The clear solution was concentrated to remove most solvent then keep in room temperature over night. White crystals will be formed and they were filtered out. The needle-like crystal will be dried in an oven at 80-100° C. 9-DHAB was obtained as white needles, purity large than 98%.

### 2. Protection of 9-DHAB

### First Alternative

2.1: 10 Grams of 9-DHAB was dissolved in 100 ml of CH₂Cl₂, and stirred at room temperature for 5 minutes then 1.5 mole tetrabutylammonium iodide, and 5 mole acetyl chloride were added, the mixture was stirred at room temperature for 8 hours or until the reaction was completed (checked by TLC). After the reaction was completed, 300 ml of water was added to stop the reaction. The mixture was extracted with 200 ml of CH₂Cl₂, and the organic layer was collected and concentrated under vacuum until dryness. The residue was purified by flash column chromatography on silica gel, eluting with a mixture of hexane: ethyl acetate (4:6) to yield 7,13-diacetyl-9-dihydrobaccatin III. Yield: >90%

### Second Alternative

2.2: 10 Grams of 9-DHAB was dissolved in 100 ml of CH₂Cl₂, and stirred at room temperature for 5 minutes then 3-5 mole-acetic anhydrite and 2 mole of 4-dimethylaminopyridine (DMAP) were added, the mixture was stirred at room temperature until the reaction was completed (checked by TLC). Workout as above obtained 7,13-diacetyl-9-dihydrobaccatin III as white crystals. Yield: 100%.

### Third Alternatives

2.3: Alternative procedures for the protection of the 7-hydroxyl group of 9-DHAB include reaction with halogen-substituted acetic anhydrite, acetyl chloride, halogen-substituted acetyl chloride, acetyl bromide, a methoxybenzyl group, a tosyl group, a substituted benzyl group, dihydropyran, benzylformate, substituted benzylformate, methoxymethyl group, a benzoylmethyl group or a substituted benzoylmethyl group.

3. Oxidation of 7,13-diacetyl-9-dihydrobaccatin III.

10 Grams of 7,13-diacetyl-9-dihydrobaccatin III was placed in to 250 ml round bottom flask Then 1.5 mole of 4-methylmorpholine N-oxide was added. The mixture was dissolved in 100 ml of dichloromethane or acetonitrile and 5% (v/v) of 4 A molecular sieve was added. The mixture was stirred at room temperature (about 25° C.) for about 10 minutes, following which 0.05 mole of the oxidizing agent tetra-n-propylammonium perruthenate (TPAP) was added. The mixture was stirred overnight at room temperature or until the reaction was completed, then was poured through a short silica gel column, eluting with dichloromethane. The dichloromethane portion was concentrated to dryness. The residue was purified by flash column chromatography on silica gel, eluting with a mixture of hexane and ethyl acetate (4:6) to yield 7,13-diacetylbaccatin III as white crystals. Yield: >90%.

4. 10-deacetylbaccatin III (10-DAB)

### First Alternative

4.1: 5 Grams of 7-chloroacetylbaccatin III or 7-acetylbaccatin III was dissolved in 150 ml of ethanol, and 3 mole equivalent hydrazine hydrate was added and the mixture was stirred at room temperature for 2 hours, checked by TLC, or until the reaction was completed. By means of a workup as described above, 10-deacetylbaccatin III was obtained as white crystals. Yield: >75%

### Second Alternative

### 4.2: Preparation of 7-Acetylbaccatin III

To a solution of 7,13-diacetyl-9-dihydrobaccatin III (10 g) in 200 ml of tetrahydrofuran at −45 C. was added methyllithium (1.4 M in ether, 6 equiv) or n-butyllithium (1.6 M in ether, 4-6 equiv) over 10 minutes, and the deacetylation was followed by TLC until completion. This mixture was quenched by pouring into buffer and extracted with ethyl acetate (EtOAc). The organic layer was washed with brine and then the solvent was evaporated. The residue was purified by flash chromatography using hexane: ethyl acetate (1:1) to obtain 7-acetylbaccatin III as a white solid. Yield: >70%.

4.3: Preparation of 10-deacetylbaccatin III

5 Grams of 7-acetylbaccatin III was dissolved in 100 ml of tetrahydrofuran or dichloromethane, and 1 mole equivalent sodium methoxide (CH₃ONa) was added. The mixture was stirred at room temperature for 2 hours, checked by TLC, or until the reaction was completed. By means of a workup as described above, 10-deacetylbaccatin III was obtained as white crystals. Yield: >75%.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A process for the conversion of 9-dihydro-13-acetylbaccatin to 10-deacetylbaccatin III, comprising the steps of:
a) protecting said 7-hydroxl group of 9-dihydro-13-acetylbaccatin and converting the 7-hydroxyl-protected 9-dihydro-13-acetylbaccatin to 7,13-diacetyl-9-dihydrobaccatin III.;
b) reacting said 7,13-diacetyl-9-dihydrobaccatin III with 4-methylmorpholine N-oxide in a suitable solvent and oxidizing the reaction product to yield 7,13-diacetylbaccatin III;
c) deacetylating said 7,13-diacetyl-9-dihydrobaccatin III to yield 7-acetylbaccatin III; and
d) converting said 7-acetylbaccatin III to 10-deacetylbaccatin III.

2. A process for the conversion of 9-dihydro-13-acetylbaccatin to 10-deacetylbaccatin III, comprising the steps of:
a) reacting 9-dihydro-1 3-acetylbaccatin with tetrabutylammonium iodide and acetyl chloride in a suitable solvent to yield 7,13-diacetyl-9-dihydrobaccatin III;
b) reacting said 7,13-diacetyl-9-dihydrobaccatin III with 4-methylmorpholine N-oxide in a suitable solvent and oxidizing said reaction product to yield 7,13-diacetylbaccatin III.;
c) deacetylating said 7,13-diacetyl-9-dihydrobaccatin III to yield 7-acetylbaccatin III; and
d) converting said 7-acetylbaccatin III to 10-deacetylbaccatin III.

3. The process as claimed in claim 1, wherein, in step a) said 7-hydroxl group of 9-dihydro-13-acetylbaccatin is protected by reaction with a compound which is selected from the group consisting of acetic anhydrite, halogen-substituted acetic anhydrite, halogen-substituted acetyl chloride, acetyl bromide, a methoxybenzyl group, a tosyl group, a substituted benzyl group, dihydropyran, benzylformate, a substituted benzylformate, a methoxymethyl group, benzoylmethyl and a substituted benzoylmethyl.

4. The process as claimed in claim 3, wherein said 7-hydroxl group of 9-dihydro-13-acetylbaccatin is protected by reaction with acetic anhydrite.

5. The process as claimed in claim 2, wherein said suitable solvent is dichloromethane.

6. The process as claimed in any one of claims 1 to 5, wherein, in step b) said oxidizing of said reaction product of 7,13-diacetyl-9-dihydrobaccatin III with 4-methylmorpholine N-oxide in a suitable solvent is effected with an oxidizing agent which is selected from the group consisting of tetra-n-propylammonium perruthenate, Collin's reagent and activated methyl sulfoxide.

7. The process as claimed in claim 6, wherein said oxidizing agent is tetra-n-propylammonium perruthenate.

8. The process as claimed in claim 6 or claim 7, wherein said suitable solvent is dichloromethane or acetonitrile.

9. The process as claimed in any one of claims 1 to 8, wherein, in step c) said deacetylating of said 7,13-diacetyl-9-dihydrobaccatin III to yield 7-acetylbaccatin III is effected with methyllithium in an ether solvent or with butyllithium in an ether solvent.

10. The process as claimed in any one of claims 1 to 9, wherein, in said step d), the converting of said 7-acetylbaccatin III to 10-deacetylbaccatin III is effected by reaction with hydrazine hydrate in a suitable solvent.

11. The process as claimed in claim 10, wherein said suitable solvent is ethanol.

12. The process as claimed in any one of claims 1 to 11, wherein, in step d), the converting of said 7-acetylbaccatin III to 10-deacetylbaccatin III is effected with an alkali metal methoxide in a suitable solvent.

13. The process as claimed in claim 12, wherein said alkali metal methoxide is sodium methoxide.

14. The process as claimed in claim 12 or 13, wherein said suitable solvent is tetrahydrofuran or dichloromethane.

15. A process for the conversion of 9-dihydro-13-acetylbaccatin to 10-deacetylbaccatin III, comprising reacting 7-chloroacetylbaccatin with hydrazine hydrate in a suitable solvent.

16. The process as claimed in claim 15, wherein said suitable solvent is ethanol.
